(19) United States
(12) Patent Application Publication
(10) Pub. No.: US 2025/0172565 A1
(43) Pub. Date: May 29, 2025

(54) A MAGNETIC MICRO-NEEDLE TO ISOLATE SINGLE IMMUNOMAGNETICALLY LABELED CELLS

(71) Applicant: Universiteit Twente, Enschede (NL)

(72) Inventors: Michiel Stevens, Enschede (NL); Philip Harder, Enschede (NL); Leonardus Wendelinus Mathias Marie Terstappen, Enschede (NL)

(21) Appl. No.: 18/843,491

(22) PCT Filed: Mar. 23, 2023
(86) PCT No.: PCT/EP2023/057517

§ 371 Date: Sep. 3, 2024

Related U.S. Application Data
(60) Provisional application No. 63/322,822, filed on Mar. 23, 2022.

(51) Int. Cl. G01N 33/58; C12M 1/26; C12Q 1/6886

(52) U.S. Cl. CPC G01N 33/585; C12M 33/04; C12Q 1/6886; G01N 33/582; C12Q 2600/156

(57) ABSTRACT

A device and methods for a magnetic micro-needle enables the selection of immunomagnetically labelled target cells. By changing the magnetic strength of the needle the speed and selectivity of the cell, selection can be adapted thus differentiating between cells with different amounts of magnetic labelled antigens on the surface of the target cells. The magnetic micro-needle thus makes use of the same cell property as used for their enrichment from the bodily fluid and identification of the target cells by their immunofluorescence profile to allow the use of the same platform avoiding any need to transfer the immunomagnetically enriched sample before selection of the cells by the magnetic micro-needle, making this device ideally suited for subsequent detailed interrogation.

### CROSS REFERENCE TO RELATED APPLICATIONS

This application is the National Stage of International Application No. PCT/EP2023/057517, Mar. 23, 2023 filed, which claims the benefit of and priority to U.S. Provisional Application No. 63/322,822, filed 23 Mar. 2022, where permissible incorporated by reference in its entirety.

### BACKGROUND

### Field of the Invention

The present invention relates to a method for immunomagnetic manipulation of rare cells using a magnetic needle to pick and place single magnetic particles that may be associated with such cells, as for example, Circulating Tumor Cells (CTC) or clusters of cells manipulated using an electromagnetic needle.

### Description of Related Art

The isolation of intact single cells is an important tool in biological research. For those applications where an abundance of cells is available often techniques as fluorescence activated cell sorting (FACS) or limited dilution are used. However, when cells are rare, such as Circulating Tumor Cells (CTC), there is an increased appreciation on the information that can be attained from single, intact cells. For this purpose multiple commercially available single cell isolation techniques exist, including the FACS and laser dissection approach as well as more recently developed techniques as the CellCelector, DEP-Array and Puncher systems. Additionally, multiple research groups have shown proof of principle work on chip based single cell isolation and/or direct characterization strategies. While some of these technologies can work with thousands to millions of cells as inputs, in the case of rare cells an initial enrichment step is needed in order to achieve a sufficient sample purity. Immunomagnetic enrichment by the FDA cleared CellSearch system is frequently used for enrichment, after which the immunomagnetically enriched and fluorescently labelled cells are obtained from the cartridges. The loss of cells during sample transfer and the follow-up isolation procedure is however a serious constraint for samples containing only few CTC.

The key element in these positive immunomagnetic enrichment procedures is the binding of a magnetic label to the cells of interest. As a result, the cells can be manipulated using magnetic fields. This characteristic is mostly used to hold all the labeled cells in place using large external magnetic configurations, allowing for the separation of bound cells from unbound cells, or to remove liquid during staining procedures. When however a local magnetic field is used, individually magnetic particles can be selectively manipulated, as shown by Timonen and Grzybowski, who used a small electromagnetic needle in order to pick and place single magnetic particles [12]. Similarly, local magnetic fields have been used to selectively attract magnetic beads bound to cells as a tool to measure cell flexibility [13]. Moving single cells by using magnetic clusters manipulated by external magnetic fields has also been done [14]. FACS, DEPArray and self-sorting microchips do not make use of the magnetic label present on the target cells and require sample transfer prior to single cell isolation. Here, we show the use a of a magnetized stainless steel pin attached to a fluorescent microscope used for the identification of the cells to directly pick-up and transfer the immunomagnetically labelled and fluorescently labelled circulating tumor cells thereby reducing cell loss and avoiding the use of yet another platform to select individual cells.

### BRIEF SUMMARY OF THE INVENTION

Immunomagnetic enrichment of cell populations from bodily fluids followed by immunofluorescent labelling is an established sample preparation method for the detection and enumeration of rare cells. For detailed analysis of the content of these rare cells, the sample needs to consist solely of the rare cells and when heterogeneity within the cell population needs to be determined the cells need to be retrieved individually. Although several technologies are available to obtain 100% pure cells either individually or in bulk all suffer from cell losses which are either inherent to the technology or the sample transfer from one platform to the other. The present invention provides for a device and methods for a magnetic micro-needle which enables the selection of the immunomagnetically labelled target cells. Moreover, by changing the magnetic strength of the needle the speed and selectivity of the cell selection can be adapted thus differentiating between cells with different amounts of magnetic label and antigens on the surface of the target cells. The magnetic micro-needle thus makes use of the same cell properties used for their enrichment from the bodily fluid and identification of the target cells by their immunofluorescence profile to allow the use of the same platform, avoiding any need to transfer the immunomagnetically enriched sample before selection of the cells by the magnetic micro-needle.

### BRIEF DESCRIPTION OF THE FIGURES

Some embodiments of the present invention are illustrated as an example and are not limited by the figures of the accompanying drawings:

FIG. 1 (A) Schematic drawing of transfer steps and B) Image of the used PDMS structure in the magnetic transfer system.

FIG. 2 shows the basic magnetic needle cell pick and placement with an example of selective transfer and repositioning of magnetically labeled tumor cells, where 4 magnetically labeled cells are removed from one position and placed into a line at a second position.

FIG. 3 A Top view with B Side view of the controlled magnetic retraction system allowing the transfer of single cells to another receptacle.

FIG. 4 A) Overview of a 30-well microscope slide used to place single cells onto. B) Overview image of a single cell showing a single LnCAP cell transferred to the microwell using the magnetic needle and retraction system. C) Zoom in of the isolated cell.

FIG. 5 A) Top view and B) isometric view of the electromagnetic coil designed to fit around the magnetic needle retraction system. C) the schematic representation of the coil windings.

FIG. 6 Pickup success rate of magnetically labeled LnCAP cells using a needle magnetized using increasing voltages applied to the used electromagnetic coil.

FIG. 7 Average transfer time (A) and success rate (B) of a de-magnetized and magnetized pin manipulating magnetic LNCaP, magnetic PC3-9 and nonmagnetic lymphocytes. A clear difference between lymphocytes and CTCs is visible, that might be caused by the size, the magnetic particles or the fixation of the CTCs.

FIG. 8 Transfer time (A), Success rate (B), Attachment time (C) and Release time (D) for the transfer of magnetically labelled and unlabelled LNCaP and PC3-9 cells using a magnetized pin, showing a higher success rate, lower transfer time, attachment and release attempts for LnCAP as well as if the cell has magnetic particles bound to it. Remarkable is the extraordinarily high value of release attempts for unlabelled PC3-9 cells, possibly due to another mechanism, based on the applied pressure between the pin, the cell, and the deposition surface.

FIG. 9 (A) Average transfer times of ten cells manipulated at increasing number of cells per cavity showing an increase in time needed for transfer with increasing concentration. (B) Success rates at increasing number of cells per cavity. A plain fall off is visible in successful transfer acts, starting from 100% at ten thousand cells per cavity reaching 0% at 160 thousand cells per cavity. (C) Average attachments in concentrations increases showing a decrease of attachment attempts with increasing cell concentration and (D) release attempts with increasing cell concentration. Time and attempts are calculated over the successful transfers, resulting in no value for the 160 thousand cell per cavity.

FIG. 10 Schematic, 1A-3A and photos 1B-3B, of the retraction principle to move a cell on a magnetic needle from one container to the other by means of a movable fluid reservoir. The cell is attached with all shown elements submerged in the sample liquid, panel 1A-1B, next, the holder containing the needle is retracted to create a separate reservoir that holds the cell in liquid while contact with the sample reservoir is removed, panel 2A-2B. Finally, the syringe is brought into contact with the target location or volume and the needle and holder are protruded in order to facilitate cell release, panel 3A-3B.

FIG. 11 Schematic representation of the isolation of CTC through DLA, the immunomagnetic enrichment of CTC, the fluorescent labeling and detection of CTC followed by the selection and transfer of the CTC for DNA analysis and molecular confirmation.

FIG. 12 Schematic representation of the selective magnetic single cell transfer with a few example applications.

FIG. 13 shows the DNA quality control results, showing a maximal genomic integrity index (Gii) score of 4 for all single CTC. Of the three PC3-9 cells, two show a maximal Gii score, while the amplification of one of the PC3-9 cells shows a Gii of 2.

### DETAILED DESCRIPTION OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “compromises” and/or “compromising,” when used in this specification, specify the presence of stated, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those used in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will also be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and claims.

The device of the present invention incorporates the use of a magnetic micro-needle as a method for cell transfer and analysis. In one embodiment, immunomagnetically enriched cells are placed on a glass surface and allowed to settle. A fluid connection is established at a sample and target location. Target cells and non-target cells are identified microscopically to allow a magnetized stainless-steel pin to be positioned above the target cell. Upon lowering the needle to the proximity of the cell, the cell attaches to the pin. This allows the cell to be lifted from the surface and remain within the solution for transfer to a target location. The cell is then positioned at the target location for release. To release the cell, the pin is moved quickly which allows the inertial, drag and surface adhesion forces to overcome the magnetic and adhesion force of the pin and settle in a static resting position. Finally, the cell may be extracted from the target location for subsequent interrogation or analysis using any means commonly available which can include a pipetting means.

One embodiment of the present invention is associated with an analysis and interrogation of prostate cancer cell line PC3-9 according to the following procedure.

### Culture Cells

The amount of magnetic particles on a cell is dependent on the number of target antigens present. The more target antigens are able to bind to the magnetic particles, the more force a magnetic field gradient will exert. For this reason two cell types with different amounts of the target antigen EpCAM were used. Cells from the prostate cancer cell line PC3-9 with a mean of 43.200 EpCAM antigens and cells from the prostate cancer cell line LNCaP with a mean of 1.868.700 EpCAM antigens. Cells were cultured in RPMI1640 (Lonza, Basel, Switzerland) supplemented with 10% FBS (Sigma-Aldrich, St. Louis, MO, USA) and 1% penicillin/streptomycin (Lonza, Basel, Switzerland). Upon reaching 70-80% confluence they were trypsinized using 0.05% trypsin-EDTA (Gibco, Waltham, MA, USA) and fixated using either CellSave (Menarini, Bologna, Italy) fixative or 1% formaldehyde. To measure the level of EpCAM expression the cells were stained using anti-EpCAM (Vu1d9)-PE (Sigma-Aldrich, St. Louis, MO, USA). The PE intensity was then measured and quantified using flowcytometry (BD FACS Aria II) and the BD Quantibrite™ Beads PE Fluorescence Quantitation Kit (BD, Franklin Lakes, NJ, USA).

### Magnetic Labeling

To label the tumor cells magnetically, the cells were incubated using either CellSearch ferrofluid (Menarini, Bologna, Italy) directed against EpCAM or first labeled with the anti-EpCAM antibody Vu1D9 coupled to biotin and subsequently incubated magnetically using Dynal C1 streptavidin beads (Thermofisher).

### Manipulation And Setup

Our cell identification setup consists of an inverted fluorescence microscope (Nikon, Japan) with a controllable X,Y Z stage (ASI), a LED light source (Lumencor) and CCD camera (Hamamatsu, Japan). In order to move the magnetic needle into proximity of the cell of interest and subsequently transferring it to another position, the needle is mounted onto a XYZ-micromanipulator (Eppendorf, Hamburg, Germany), which is attached to the microscope. To attach and position the stainless-steel pin to the micromanipulator a flexible adapter was designed and 3D printed.

### Sample Holder

To manipulate the cell, the sample is placed into a container that allows the pin to be positioned close to the cell. The sample is placed into a sample holder, consisting of a microscope slide onto which a PDMS structure is placed. The PDMS structure has nine openings in a three by three structure. Each measures five times five mm. Each of the nine resulting chambers can hold 50 μL liquid and allow for the cells being placed directly onto the glass slide. Prior to assembly the glass slide is incubated using PBS BSA 1% to prevent non-specific adhesion. After the addition of 50 μl of sample containing the cells of interest into one of sample chambers, cells are allowed to sediment onto the glass slide. Next, either PBS or cell culture medium is placed into the middle and lower chamber. The chambers are then connected by the addition of additional liquid, spanning the lower walls between the chambers, resulting in a fluid bridge.

### Stainless Steel Pins

Although any ferro-, ferri- or even (super-)para-magnetic material would be possible, for our experiments we used pins made of ferritic stainless-steel (Austerlitz INSECT PINS, Slavkov u Brna, Czech Republik), normally used for the preparation of insect displays. They are 12 mm long and 100 μm in diameter, with one of the ends being sharpened into a 12.5 μm diameter tip end.

### Magnetization of Pins

The pins can be magnetized either by permanent or electromagnets, either before starting the experiment or at the time of pickup. In the latter case the electro-magnet is placed in close proximity of the pin within the setup. When an electromagnet is used, the electromagnetic field can also be used to demagnetize the pin at the time of release. If a superparamagnetic material is used demagnetization is not needed. In our experiments we magnetized the pins prior to use, by placing them in between opposing permanent magnets (N45 magnets, Supermagnete, Germany). The maximum remnant magnetism of the used pins was determined to be 14.5 μAm2 by measuring the magnetization curve of a bundle of 10 pins using VSM. In proof of principle experiments, the needles were fitted into 22ga×0.25 in Luer dispensing tips (Instech, Plymouth Meeting, US) for easy handling and attachment into the manipulation system.

### Attachment and Release Forces

The magnetic force exerted onto the cell (Fm is determined by the magnetic moment of the magnetic particle (mp), the amount of magnetic particles (N p) and the magnetic field gradient (V̅(B)) using:

F_m=N_p m_p V̅(B) (1)

In order to be able to lift the cell upward of the surface towards the needle, this magnetic force will need to overcome at least the gravitational force, given by:

F_g=g*V_p(ρ_c−ρ_f) (2)

where g=9.81 m/s2 and, ρc=1070 kg/m3 and ρf=1000 kg/m3 are the density of the cell and fluid.

As the cell is on a surface, also any electrostatic surface adhesion forces will need to be overcome. The magnitudes of these forces are dependent on the membrane characteristics as well as the surface properties of the used material. Using blocking and coating buffers these forces will need to be minimized in order to allow cell transfer. If no magnetic label is present or the magnetic force is insufficient, the needle can be brought into contact with the cell, creating an adhesion force between the cell and the needle. If needed, the cell can be pushed sideways, dislodging it from the surface, thereby removing the existing adhesion forces to the surface. Once the cell has been attached to the needle, the surface adhesion force in combination with any remaining magnetic force will hold the cell in place on the pin. For removal, this force will need to be toppled using (a combination of) acceleration, drag and surface attachment forces. The force that acts on the cell as the needle is rapidly accelerated is by the mass of the cell (m) in combination with the acceleration.

F_acc=ma (4)

The drag force on the cell when moving it through a liquid in the laminar regime is given by Stokes' law as a function of the viscosity η(Pa·s), particle radius r (m) and velocity of the particle vp and fluid vf (m/s) as follows:

$$F\_d = 6 πη r ( v\_p - v\_r ) \qquad ( 3 )$$

The surface adhesion force is dependent on the surface, cell and medium properties. In general it depends on the application whether or not this force is a useful addition. If the cell is to be removed from the target location after placement, this force should be kept to a minimum. If the cell is to remain at the target location additional binding to the surface can be facilitated by using either specific or non-specific coatings.

### Efficiency

To show the efficiency of the described method, we picked up 20 randomly selected cells and placed these onto a newly selected location. The time and number of attempts for pickup, transfer and placement were noted, and the cell transfer was called successful if all three steps could be performed in under two minutes.

### Results

### Principle of Magnetic Micro-Needle Cell Selection

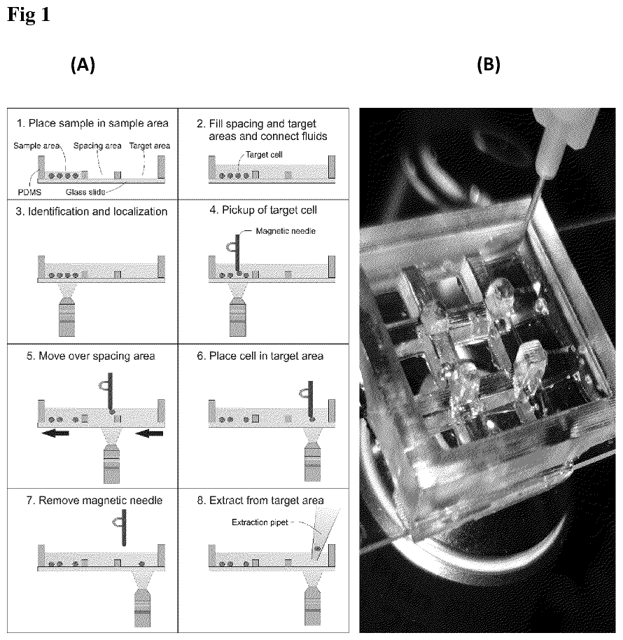

FIG. 1A depicts the principle of magnetic micro-needle cell transfer as performed in our proof of principle experiments. The immunomagnetically enriched cells are placed on a glass surface and allowed to sediment in step 1. Next, a fluid connection is established to the target area, possibly via a spacing area separating the sample and target areas step 2. The position of the target cells (green) and non-target cells (purple) is identified by microscopy step 3. A magnetized stainless-steel pin is positioned above the target cell and by lowering the needle is brought into proximity of the cell, causing the cell to attach to the pin step 4. Next, the cell is lifted from the surface through the solution and transferred to the target location step 5. The cell is placed on the target location (6). Then, to release the cell, the pin is moved quickly to allow the inertial-, drag- and surface adhesion forces to overcome the magnetic and adhesion force to the pin step 7. Finally, the cell is if desired extracted from the target location by pipetting out the liquid step 8. FIG. 1B provides an image of the PDMS structure used in the magnetic transfer system.

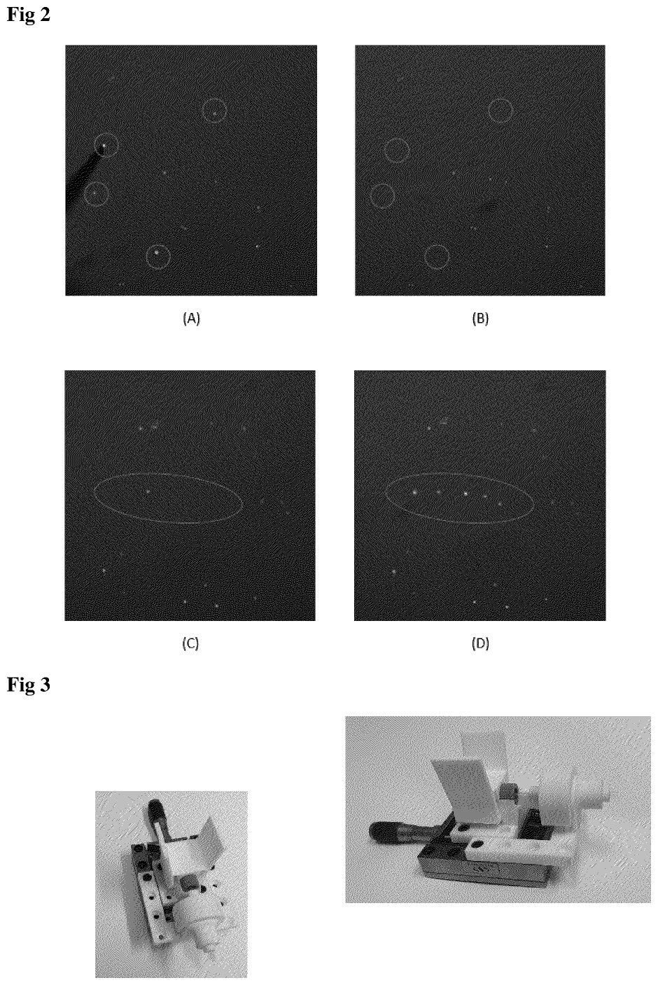

Example for the transport of four magnetically labelled LNCaP cells using a pre-magnetized magnetic needle. FIG. 2A shows the pick-up location before pick-up of the cells and FIG. 2B shows the pick-up location after the cell pick-up. FIG. 2C shows the release location of the cells before any cell was transported. FIG. 2D shows the release location after the cells were transported.

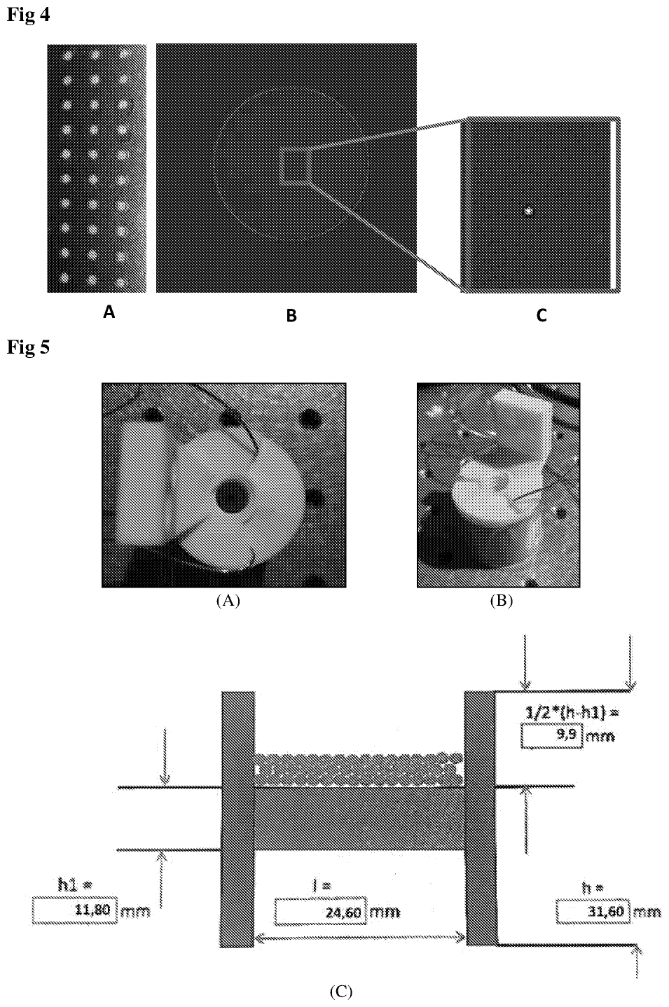

Cell retraction for the transfer to other receptacles is shown in FIG. 3. Although cell manipulation allowing the pickup and placement of single cells has many applications, often the transfer to a different container or receptacle is needed. As the retraction of the magnetic needle through the surface tension of the liquid often results in the detachment of the cell, one embodiment of the present invention is designed with a retraction holder to controllably retract the cell into a small diameter tubing, shown in FIG. 3. Using this retraction, the single cell can be transferred to a different container or receptacle without leaving the liquid, allowing as an example the placement of a single cell onto a single well of a multi-well microscope slide, as shown in FIG. 4.

One embodiment in the present invention incorporates the use of electromagnetic induced magnetization of the needles. To ensure the used needles were fully unmagnetized, the needles were first fully demagnetized. This was done by heating the needles at 800° C.

To prove the principle of using an electromagnetic coil to control the magnetization of the steel needle during the magnetic pickup, we designed an electromagnetic coil surrounding a previously designed needle retraction system, see FIG. 5. The maximal voltage that could be used for a longer time with this coil without excessive heating occurring was 8 V.

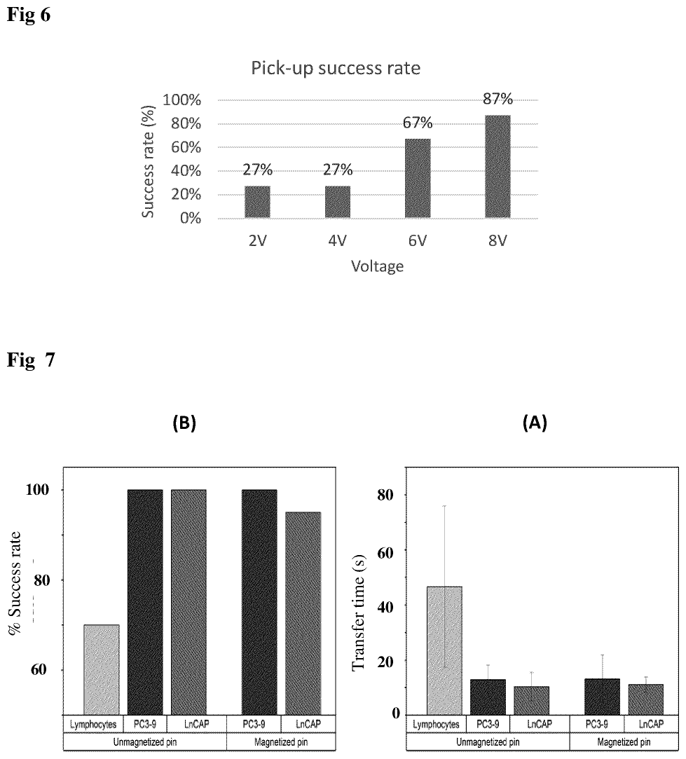

Using this system we attempted to pick up 15 magnetically labeled LnCAP cells using a magnetization voltage of 2V, 4V, 6V or 8V. Results in FIG. 6 show an increase in the pickup success indicating a controllable attachment force, which could be tuned to the magnetic moment of the target cell, which will be different for each application due to changes in cell type, targeted antigen expression and magnetic particle type used.

### Transfer Efficiency and Time Needed for Magnetic Micro-Needle Cell Selection

Selective transfer of LNCaP cells expressing EpCAM and high levels and PC3-9 cells expressing EpCAM at low levels was tested and compared to that of lymphocytes not expressing EpCAM. The cells were labelled with EpCAM biotin followed by streptavidin Dynal beads. Magnetized and demagnetized pins were used compared to the success rate of picking and placing the target cell to its location, FIG. 7A and the transfer time FIG. 7B. The success rate for both tumor cell lines was 95-100% with no difference whether or not the pin was magnetized. Strikingly, the lymphocytes despite the absence of magnetic beads could be transferred in 70% of the cases, albeit the transfer time was significantly longer.

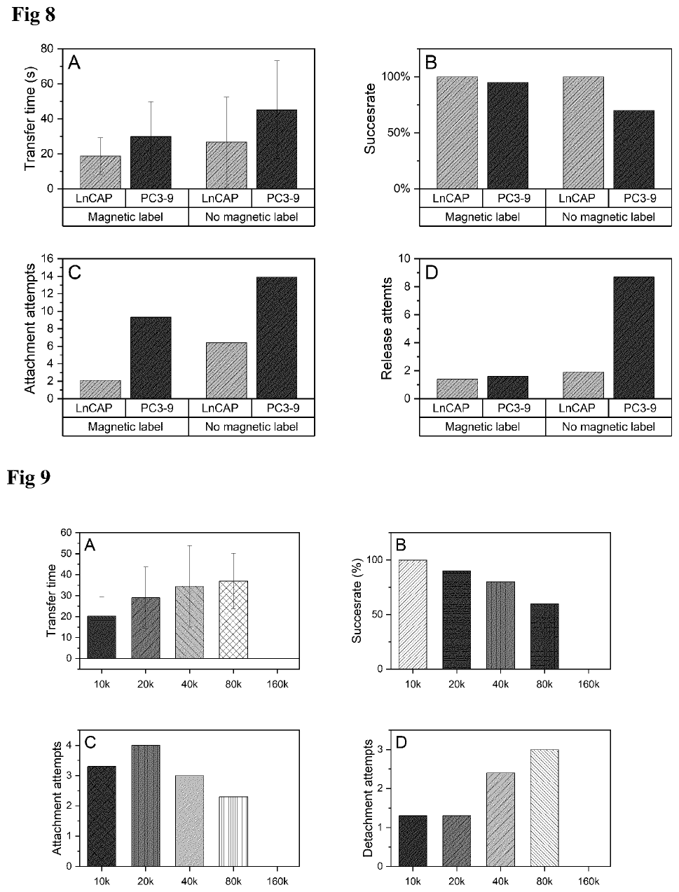

Next the success rate and transfer time the number of attempts needed to attach and release magnetically labelled and not labelled LnCAP and PC-3 cells was determined. FIG. 8A shows a success rate of 100% for both the unlabeled and the labelled LnCAP cells whereas for the magnetically labelled PC3 this was slightly less with 95% and significantly less (70%) for the non-labelled PC-3 cells. The increase in transfer time followed the amount of the magnetic label on the cells as shown in FIG. 8B which is also reflected by the number of attempts needed to pick-up the cells FIG. 8C. To release the cells the cells attached to the needle were transported to the desired location and placed on the position without alteration of the magnetization of the needle or bringing the cell to the upper surface of the liquid. The number of attempts to release the cells was largest for the non-magnetically labelled PC3 cells FIG. 8D.

As the density of the cells present in the sample will have an influence on the efficiency to selectively pick target cells among the bystander cells experiments were conducted in which magnetically labelled LnCAP cells were placed among different amounts of unlabeled PC3-9 cells. The PC3-9 cell densities tested were 104, 2×104, 4×104, 8×104 and 1.6 105 cells spread over a surface of 25 m2 (5×5 mm cavity). With an average diameter of PC3-9 cells of 16 μm this results in an occupancy of the 25 mm2 surface of 10%, 20%, 41%, 82% and 164% respectively. This means that at the highest concentration the cells are stacked on top of each other. The success rate, the transfer time, the number of attempts needed to select and release the LnCAP cells on the target location is shown in FIG. 9. Here in FIG. 9A, the average transfer times of ten cells manipulated at increasing number of cells per cavity showing an increase in time needed for transfer with increasing concentration. FIG. 9B shows the success rates at increasing number of cells per cavity. A plain fall off is visible in successful transfer acts, starting from 100% at ten thousand cells per cavity reaching 0% at 160 thousand cells per cavity. The graph in FIG. 9C shows the average attachments in concentrations increases showing a decrease of attachments attempts with increasing cell concentration. While FIG. 9D shows release attempts with increasing cell concentration. Time and attempts are calculated over the successful transfers, resulting in no value for the 160 thousand cell per cavity.

A clear relation between cell density and the effectivity of the magnetic micro-needle cell selection can be observed. The lesson learned from these experiments is that the surface area on which the immunomagnetically enriched and fluorescently labelled cells are placed needs to accommodate all cells present in the sample, with surface coverage that can reach up to 80% while still retaining an above 50% success rate. In most applications however the surface coverage should be limited to 10% or less.

### Transfer to Other Containers

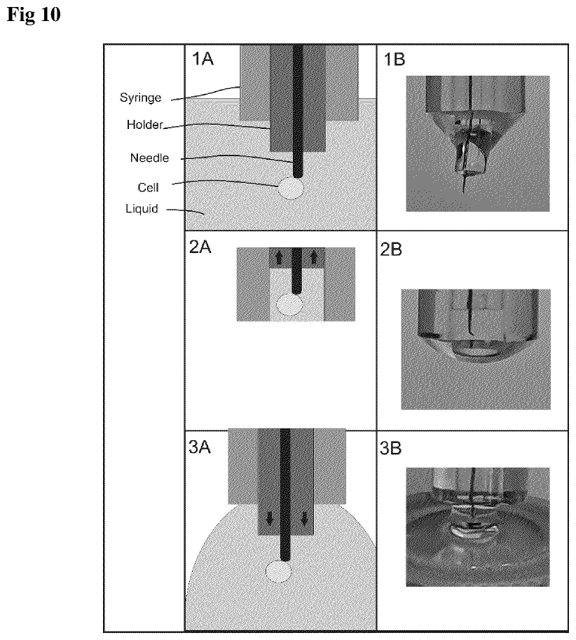

In one embodiment, transfer of the cell to a target area occurred within the same container, connected by fluid to the sample area. In this manner the cell can be transferred to any location that is connected by fluid to the sample area. In most applications however the cell needs to be placed onto or into a standard sample format. This can be achieved by pipetting the cell out as performed in our proof of principle, but this removes the possibility of precise placement of the cells. Another way of transfer without leaving the liquid is shown in FIG. 10. Here, the needle is mounted into a holder that protrudes from a syringe like container, panel 1A-1B. After picking the cell up, the magnetic needle is retracted within the syringe like chamber, panel 2A-2B. When the syringe is then removed from the sample area, the liquid within is transferred along, thereby keeping the needle and attached cell contained in the liquid. After transfer to the target area/liquid the needle is protruded again and the cell is released as described previously, 3A-3B. This way the cell can be transferred to a different type of sample holder or liquid, without the need for the cell and needle to overcome the surface tension. Additionally, the cell can be moved at large speeds without being subjected to large drag forces.

### Examples of the Use of the Magnetic Micro-Needle Cell Selection

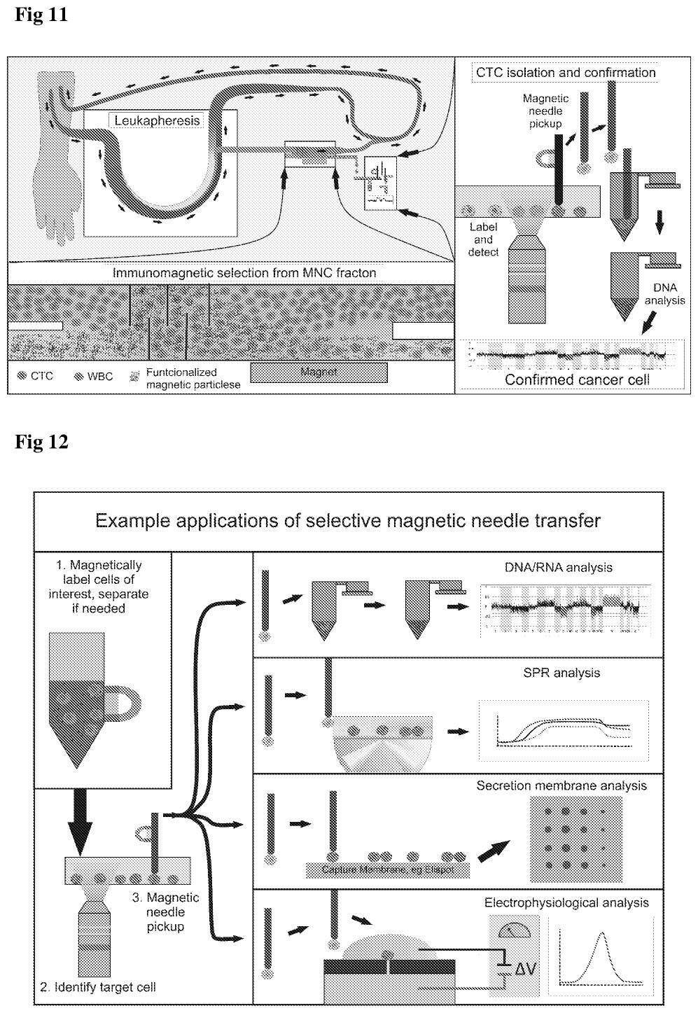

Single cell selection by means of a magnetic micro-needle is only one of the tools needed to diagnose and confirm cancer and/or guide cancer treatment using CTC. The load of tumor cells in blood is directly related to the prognosis of the cancer patient with metastatic disease [15-17] and the presence of only one or more CTC in a tube of blood in cancer patients without known dissemination of the disease identifies patients with high risk of recurrence of the cancer [18]. For the latter application a greater sensitivity and specificity of CTC detection is needed. As shown in FIG. 11 to increase the sensitivity, larger blood volumes will need to be analyzed which can be obtained through Diagnostic Leukapheresis (DLA). To increase the specificity the DNA of the cell identified as CTC can be analyzed for the presence of genetic alterations consistent with cancer. FIG. 10, panel 1A-3A, provides a series of representations with corresponding images, panel 1B-3B, depicting the retraction principle. The overall retraction principle to move a cell on a magnetic needle from one container to the other by means of a movable fluid reservoir is shown. The cell is attached with all shown elements submerged in the sample liquid, panel 1B-3B, next, the holder containing the needle is retracted to create a separate reservoir that holds the cell in liquid while contact with the sample reservoir is removed, panel 2A-2B. Finally, the syringe is brought into contact with the target location or volume and the needle and holder are protruded in order to facilitate cell release, panel 3A-3B.

A patient is subjected to DLA, FIG. 11, in which the mononuclear cell fraction is extracted and passed through an immunomagnetic selection chamber in which immunomagnetic particles targeting the CTC are added [19]. Under influence of a magnetic gradient the cells labelled with the immunomagnetic particles and the free immunomagnetic particles are separated from the unlabeled cells and placed in any receptacle that allows for staining and visualization, such as but not limited to the “Cell-Trench” all other cells can be returned to the patient. In the “Cell-Trench” reagents are added to label the cells and using a fluorescent microscope the CTC are identified among the leukocytes carried over through the procedure. The magnetic micro-needle can now be placed above a CTC candidate after which the transferred CTC can be placed in a PCR tube for DNA analysis and cancer confirmation. Subsequent candidate CTC can be either placed in individual PCR tubes or placed in the same PCR tube. A schematic representation of this process is shown in FIG. 11 with the isolation of CTC through DLA, immunomagnetic enrichment of CTC, fluorescent labeling and detection of CTC followed by selection and transfer of the CTC for DNA analysis and molecular confirmation.

Availability of CTC enables the selection of effective drugs through probing the expression of drug targets as well as the actual effectivity of the drug on the CTC. Moreover, the availability of “pure” CTC would enable the discovery of novel drugs by exploring the content of the CTC. FIG. 12 depicts the role of the magnetic micro-needle in this process with examples of selective magnetic needle transfer. After immunomagnetic enrichment of CTC from a peripheral blood and fluorescent labelling of the cells in the sample in step 1. The sample is placed in the “Cell-Trench” in step 2. After identification by fluorescent microscopy the CTC are picked up by the magnetic micro-needle, step 3, and transferred to a location for follow-up analysis having an interrogation platform. In this example a tube for content analysis (protein, RNA, DNA), an SPRi surface for real time monitoring of cell secretion products and the response after addition of drug candidates, a surface for protein capture for analysis (e.g. Elispot) and on a pore for electrophysiological analysis of the selected single cell.

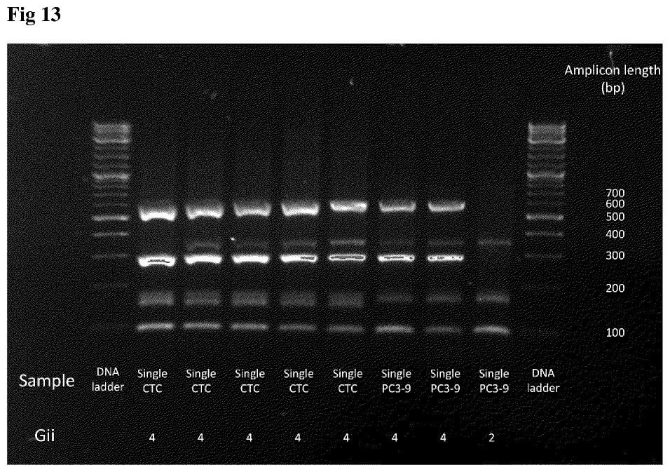

A still further embodiment of the present invention allows for its use in genomic analysis of isolated cells, FIG. 13. As the main reason for single-cell isolation is to do (epi)genomic analysis the present invention was used to test the ability to perform DNA amplification after single-cell MagNeedle isolation. For this, we isolated 5 single CTC from a prostate cancer patient sample enriched using the CellSearch system. We also isolated three magnetically labeled PC3-9 cells. FIG. 13 shows the DNA quality control results, showing a maximal genomic integrity index (Gii) score of 4 for all single CTC. Of the three PC3-9 cells, two show a maximal Gii score, while the amplification of one of the PC3-9 cells shows a Gii of 2, indicating that although the cell was successfully isolated, not all DNA is present in the resulting amplified material. This could be due to cell damage due to necrosis or apoptosis either before, during or after single cell isolation.

### REFERENCES

1. Lim S Bin, Di Lee W, Vasudevan J, Lim W-T, Lim C T. Liquid biopsy: one cell at a time. npj Precis Oncol. 2019; 3. doi:10.1038/s41698-019-0095-0
2. Wu C P, Wu P, Zhao H F, Liu W L, Li W P. Clinical Applications of and Challenges in Single-Cell Analysis of Circulating Tumor Cells. DNA Cell Biol. 2018; 37: 78-89. doi:10.1089/dna.2017.3981
3. Keller L, Pantel K. Unravelling tumour heterogeneity by single-cell profiling of circulating tumour cells. Nat Rev Cancer. 2019; 19: 553-567. doi:10.1038/s41568-019-0180-2
4. Nelep C, Eberhardt J. Automated rare single cell picking with the ALS Cellcelector™. Cytom Part A. 2018; 93: 1267-1270. doi:10.1002/cyto.a.23568
5. Di Trapani M, Manaresi N, Medoro G. DEPArray™ system: An automatic image-based sorter for isolation of pure circulating tumor cells. Cytom Part A. 2018; 93: 1260-1266. doi:10.1002/cyto.a.23687
6. Stevens M, Oomens L, Broekmaat J, Weersink J, Abali F, Swennenhuis J, et al. VyCAP's puncher technology for single cell identification, isolation, and analysis. Cytom Part A. 2018; 93: 1255-1259. doi:10.1002/cyto.a.23631
7. Pei H, Li L, Han Z, Wang Y, Tang B. Recent advances in microfluidic technologies for circulating tumor cells: Enrichment, single-cell analysis, and liquid biopsy for clinical applications. Lab Chip. 2020; 20: 3854-3875. doi:10.1039/d01c00577k
8. Song Y, Tian T, Shi Y, Liu W, Zou Y, Khajvand T, et al. Enrichment and single-cell analysis of circulating tumor cells. Chem Sci. 2017; 8: 1736-1751. doi:10.1039/c6sc04671a
9. Lamanna J, Scott E Y, Edwards H S, Chamberlain M D, Dryden M D M, Peng J, et al. Digital microfluidic isolation of single cells for -Omics. Nat Commun. 2020; 11: 1-13. doi:10.1038/s41467-020-19394-5
10. Zhang M, Zou Y, Xu X, Zhang X, Gao M, Song J, et al. Highly parallel and efficient single cell mRNA sequencing with paired picoliter chambers. Nat Commun. 2020; 11: 1-13. doi:10.1038/s41467-020-15765-0
11. Wang Y, Wang X, Pan T, Li B, Chu J. Label-free single-cell isolation enabled by microfluidic impact printing and real-time cellular recognition. Lab Chip. 2021; 21: 3695-3706. doi:10.1039/d11c00326g
12. Timonen J V I, Grzybowski B A. Tweezing of Magnetic and Non-Magnetic Objects with Magnetic Fields. Adv Mater. 2017; 29. doi:10.1002/adma.201603516
13. Cenev Z, Zhang H, Sariola V, Rahikkala A, Liu D, Santos H A, et al. Manipulating Superparamagnetic Microparticles with an Electromagnetic Needle. Adv Mater Technol. 2018; 3: 1-9. doi:10.1002/admt.201700177
14. Zhu L, Huang W, Yang F, Yin L, Liang S, Zhao W, et al. Manipulation of Single Cells Using a Ferromagnetic Nanorod Cluster Actuated by Weak A C Magnetic Fields. Adv Biosyst. 2019; 3: 1-11. doi:10.1002/adbi.201800246
15. Cristofanilli M, Hayes D F, Budd G T, Ellis M J, Stopeck A, Reuben J M, et al. Circulating tumor cells: A novel prognostic factor for newly diagnosed metastatic breast cancer. J Clin Oncol. 2005; 23: 1420-1430. doi:10.1200/JCO.2005.08.140
16. Moreno J G, Miller M C, Gross S, Allard W J, Gomella L G, Terstappen LWMM. Circulating tumor cells predict survival in patients with metastatic prostate cancer. Urology. 2005; 65: 713-718. doi:10.1016/j.urology.2004.11.006
17. Cristofanilli M. Circulating tumor cells, disease progression, and survival in metastatic breast cancer. Semin Oncol. 2006; 33: S9-14. doi:10.1053/j.seminoncol.2006.03.016
18. Van Dalum G, Stam G J, Scholten L F A, Mastboom W J B, Vermes I, Tibbe A G J, et al. Importance of circulating tumor cells in newly diagnosed colorectal cancer. Int J Oncol. 2015; 46: 1361-1368. doi:10.3892/ijo.2015.2824
19. Stevens, Michiel; Tibbe, Arjan G. J.; Broekmaat, Joska Johannes; Nijsink, Frederic Thomas; Terstappen, Leon W. M. M.; van Dallum G. Device and Method For The Continuous Trapping of Circulating Tumor Cells. United States: USPO; 20200108391, 2018.

1. A magnetic micro-needle device for selecting a target cell from a population for subsequent analysis comprising:
a) a magnetized pin;
b) an immunomagnetically labelled target cell, said cell comprising antigen with magnetic particle bound thereto;
c) a fluid reservoir container with a micromanipulator for control of the pin and cell; and
d) a cell interrogation platform,
wherein manipulation of the target cell using the magnetic pin allows for individual cell analysis.

2. The magnetic micro-needle device according to claim 1, wherein the immunomagnetically labelled target cell has a fluorescent label for identification.

3. The magnetic micro-needle device according to claim 1, wherein the reservoir container further contains a cell-trench with reagents for fluorescent labelling and microscope identification.

4. (canceled)

5. (canceled)

6. The magnetic micro-needle device according claim 33, wherein the stainless-steel pin has a length of 12 millimeters and a diameter of 100 micrometers with one end sharpened to a diameter of 12.5 micrometers and/or wherein the stainless-steel pin is magnetized by a permanent magnet or electromagnet.

7. (canceled)

8. The magnetic micro-needle device according to claim 1, having a magnetized stainless-steel pin within a cell retraction holder comprising:
a) small diameter tubing capable of retracting the pin; and
b) an electromagnetic coil to control the magnetization of the pin,
wherein the magnetized stainless-steel pin is capable of picking up an immunomagnetically enriched target cell.

9. The magnetic micro-needle device according to claim 8, wherein the small diameter tubing is a syringe.

10. The magnetic micro-needle device in claim 8, having a retraction holder comprising:
a) a syringe;
b) a holder;
c) a needle;
d) a target cell; and
e) a liquid.

11. The magnetic micro-needle device according to claim 1, wherein the immunomagnetically labelled target cells are rare cells.

12. The magnetic micro-needle device according to claim 1, wherein the reservoir container is a sample holder having a PDMS structure.

13. The magnetic micro-needle device according to claim 12, wherein the PDMS structure has nine openings in a three by three structure with each opening five by five millimeters capable of holding 50 microliters of fluid.

14. The magnetic micro-needle device according to claim 1, wherein the immunomagnetically labelled target cell has a magnetic label bound to a cell antigen.

15. The magnetic micro-needle device according to claim 1, wherein the target cell is a circulating tumor cell.

16. The magnetic micro-needle device according to claim 1, wherein the target cell is an LNCaP cell or PC3-9 cell.

17. The magnetic micro-needle device according to claim 1, wherein the immunomagnetically labelled target cell contains an anti EpCAm antibody Vu1D9 coupled to biotin and Dynal C1 streptavidin beads.

18. The magnetic micro-needle device according to claim 1, wherein the interrogation platform is selected from a group consisting of DNA/RNA analysis, SPR analysis, Secretion membrane analysis, electrophysical analysis, and a combination thereof.

19. A method for selecting an immunomagnetic target cell from a sample population for subsequent analysis comprising:
a) immunomagnetically labelling target cells in a sample population;
b) identifying a target cell, to obtain target cells comprising antigen with magnetic particle bound thereto;
c) positioning a magnetized stainless-steel pin in close proximity to the target cell to allow the cell to become attached to the pin;
d) transferring the target cell to a fluid reservoir; and
e) releasing the target cell from the pin for analysis.

20. The method for selecting a target cell from a sample population according to claim 19, wherein identifying further includes fluorescent labelling and identifying with a fluorescent microscope.

21. The method for selecting a target cell from a sample population according to claim 19, wherein the immunomagnetic target cells are rare cells.

22. The method for selecting a target cell from a sample population according to claim 19, wherein the immunomagnetic target cells express an EpCAM antigen.

23. The method for selecting a target cell from a sample population according to claim 19, wherein the immunomagnetic target cells are LNCaP cells or PC3-9 cells.

24. The method for selecting a target cell from a sample population according to claim 19, wherein the immunomagnetic target cells are labelled with anti EpCAm antibody Vu1D9 coupled to biotin and Dynal C1 streptavidin beads.

25. The method for selecting a target cell from a sample population according to claim 19, wherein the magnetized stainless-steel pin is positioned in close proximity to an electromagnet or permanent magnet for attachment of the target cell.

26. The method for selecting a target cell from a sample population according to claim 19, wherein transferring is to a moveable fluid reservoir.

27. The method for selecting a target cell from a sample population according to claim 19, wherein transferring is to a cell-trench.

28. The method for selecting a target cell from a sample population according to claim 19, wherein releasing is for the analysis from a group consisting of DNA/RNA analysis, SPR analysis, Secretion membrane analysis, electrophysical analysis, and a combination thereof.

29. A method for determining a cancer cell by genomic analysis in a subject comprising:
a) subjecting the subject to DLA, to obtain whole blood, or any other body fluid;
b) labelling CTC's with immunomagnetic particles, to obtain CTC's comprising antigens with immunomagnetic particles bound thereto;
c) separating the unlabeled cells in a cell trench for returning to the subject;
d) identifying fluorescent CTC's with a fluorescent microscope; and
e) capturing a CTC candidate with a magnetic micro-needle for DNA analysis and cancer confirmation.
wherein genomic analysis of the CTC determines the cancer cell in the subject.

30. The magnetic micro-needle device according to 1, wherein the magnetic particles allow for attaching the immunomagnetically labelled target cell to the magnetized pin upon positioning said magnetized stainless-steel pin to the proximity of said cell.

31. The magnetic micro-needle device according to claim 1, wherein the immunomagnetically labelled target cell is placed and settled on a surface.

32. The magnetic micro-needle device according to claim 1, wherein the target cell is from a cell population obtained from bodily fluid.

33. The magnetic micro-needle device according to claim 1, wherein the magnetized pin is made from ferro-, ferri- or (super-)para-magnetic material and/or wherein the magnetized pin is a magnetized stainless steel pin, preferably ferritic stainless-steel.

34. The method according to claim 19, wherein the magnetic particles allow for attaching the immunomagnetically labelled target cell to the magnetized pin upon lowering said magnetized pin to the proximity of said cell.

35. The method according to claim 44, wherein in a) the obtained immunomagnetically labelled target cell are placed and allowed to settle on a glass surface.

36. The method according to claim 19, wherein the immunomagnetic target cell is from a sample cell population obtained from bodily fluid.

37. The method according to claim 29, wherein in b) the immunomagnetic particles allow for attaching the CTC's to the magnetic micro-needle upon lowering said magnetic micro-needle to the proximity of said CTC.

38. The method according to claim 29, wherein in b) the obtained CTC's are placed and allowed to settle on a glass surface.

39. The method according to claim 29, wherein the CTC's are obtained from said DLA, whole blood, or any other body fluid of said subject.
